(19) United States
(12) Patent Application Publication
(10) Pub. No.: US 2013/0087142 A1
(43) Pub. Date: Apr. 11, 2013

(54) METERED DOSE INHALER

(71) Applicants: Prashant Kane, Baroda (IN); Satish Madhukar Gokhale, Pune (IN); Nachiket Gole, Pune (IN)

(72) Inventors: Prashant Kane, Baroda (IN); Satish Madhukar Gokhale, Pune (IN); Nachiket Gole, Pune (IN)

(73) Assignee: SUN PHARMA ADVANCED RESEARCH COMPANY LTD., Mumbai (IN)

(21) Appl. No.: 13/635,042

(22) PCT Filed: Mar. 17, 2010
(86) PCT No.: PCT/IN2011/000178

§ 371 Date: Nov. 29, 2012

(30) Foreign Application Priority Data
Mar. 17, 2010 (IN) 704/MUM/2010

(51) Int. Cl. A61M 15/00

(52) U.S. Cl. CPC A61M 15/009
USPC 128/200.23

(57) ABSTRACT

The present invention relates to metered dose inhaler devices for the administration of medicament to patients for inhalation therapy. Particularly, it discloses a metered dose inhaler device for improving medicament compliance by patients.

The present invention relates to metered dose inhaler devices for the administration of medicament to patients for inhalation therapy. Particularly, it discloses a metered dose inhaler device for improving medicament compliance by patients.

### BACKGROUND OF THE INVENTION

Inhalation has long been known as a method of administering medicines for distribution and absorption into the airways, lungs, and nasal passages. Inhalation devices are the predominant medicament delivery system for treating conditions such as asthma and other respiratory disorders such as chronic bronchitis. Proper use of an inhaler requires (1) correct assembly of the parts, (2) shaking of the inhaler before use, (3) slow exhalation of the tidal volume, (4) correct positioning of the inhaler at the mouth or nose, (4) activation immediately after initiation of the inhalation, (5) slow and deep inhalation and 7) breath holding for 6 to 10 seconds. Of the problems reported, two of the most significant are not shaking the inhaler prior to use and improper orientation of the device while inhalation. Since the active medicament and aerosol propellant are frequently in suspension, it is necessary to insure that settling of the aerosol components does not result in an improper dose of the active medication. This is particularly true in cases where the inhaler is not used for days at a time. The second factor of concern is proper vertical orientation of the inhaler and the patients head. As with most propellant systems, if the inhaler is not properly oriented when activated the use may dispense an improper mix of propellant and active medicament, resulting in improper dosing. Further, during inhalation, improper orientation of the inhaler may also result in impaction of the medication of the tongue, roof of the mouth, side of the mouth, sides of the nose, or throat. Improper orientation of the user's head in relation to the orientation of the inhaler is also problematic. When a person's head is tilted forward, human physiology is such that the airway to and from the lungs becomes more constricted, significantly restricting the flow of air to and from the lungs. (U.S. Pat. No. 6, 957,125).

U.S. Pat. No. 6,957,125 (hereinafter referred to as U.S. patent '125) discloses an apparatus for indicating proper orientation of an inhaler by a patient comprising: a housing for attachment to an inhaler; a visual indicator; and means for enabling movement of said visual indicator relative to said housing between a visible position within the visual field of the patient when an inhaler is held in proper orientation, and a concealed position when an inhaler is not held in the proper orientation. The '125 patent provides within the device a visual indicator which is comprised of an indicator ball confined inside a substantially horizontal tube within the inhaler housing. The indicator ball would remain out of view within the inhaler housing unless the inhaler is held in a substantially vertical orientation. Also, the '125 patent discloses a rattle within the tube producing an audible rattle upon shaking of the device. However, there is no mechanism taught in this prior art by which the inhaler gets locked and gets unlocked and functions only when the proper orientation is achieved and/ or when the inhaler is properly shaken before use.

Another prior art, U.S. Pat. No. 5,676,129 (hereinafter referred to as U.S. patent '129) discloses a drug dispensing apparatus comprising a pressurized container for a dispensable drug, said container comprising at a delivery end a metered dose valve and nozzle, said nozzle at its exit for receiving a metered dose of said drug substantially at said predetermined pressure, and a pressure sensor for detecting the changes in the pressure in said transfer passage. Particularly, the apparatus comprises a motion detector for detecting a shaking applied to said apparatus and means responsive to said motion detector for determining whether said shaking action satisfied predetermined criteria. The signal generated by the motion sensor may be input to the microprocessor for detection and processing, and for the activation of a display or audible signal to signify that the proper amount of shaking has been provided. The present invention however uses an inbuilt mechanism by which the user has to compulsorily shake the inhaler without this shaking action the inhaler would not work. Such provision is not disclosed in the U.S. patent '129 patent.

Several electronic mechanisms are disclosed for detecting the correct position of the inhaler, for example U.S. Pat. No. 6, 692,492 (U.S. patent '492) discloses a device characterized in that it includes a position sensor for detecting any predetermined orientation sensor for detecting any predetermined orientation of the device, in which said position sensor is connected to said electronic control circuit, and said electronic control circuit is adapted to trigger actuation of the device in said any predetermined orientation which is sensed by said position sensor. The device of the present invention is different in that the device includes in built mechanisms by which the device would not deliver the medicament unless the device is shaken first and is in correct position or angle while delivering the dose.

### OBJECT OF INVENTION

The object of the present invention is to provide an improved metered dose inhaler such that the inhaler does not deliver the medicament unless the user shakes the inhaler.

The object of the present invention is to provide an improved metered dose inhaler such that the inhaler does not deliver the medicament unless the user holds the inhaler in a correct position.

### SUMMARY OF THE INVENTION

The present invention provides an improved metered dose inhaler for delivery of preparation in the form of an aerosol comprising,
1. canister containing a medicament
2. actuator means
3. locking means that locks the actuator means and is unlocked upon shaking the device allowing the actuator means to deliver the medicament after it has been rendered homogeneous by shaking.

DETAILS OF THE FIGURES AND THE DIAGRAMS

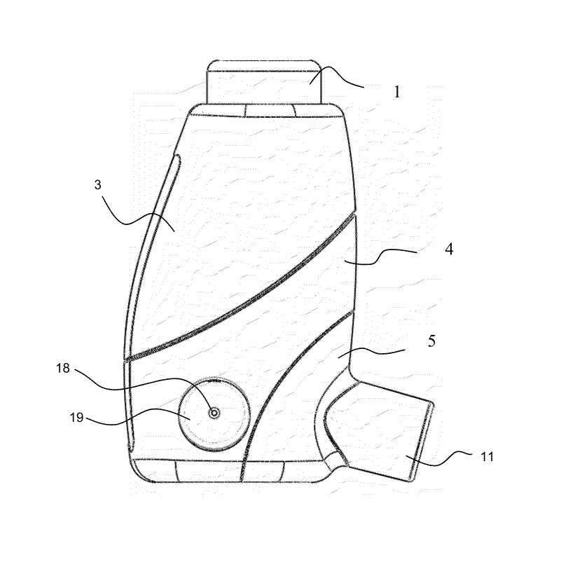

FIG. 1 is the side view of the metered dose inhaler of the present invention. 1 is canister, 3 is top body part, 4 is middle body part, 5 is removable mouthpiece, 11 is mouthpiece cap, 18 is tactile pin, 19—dose counter display.

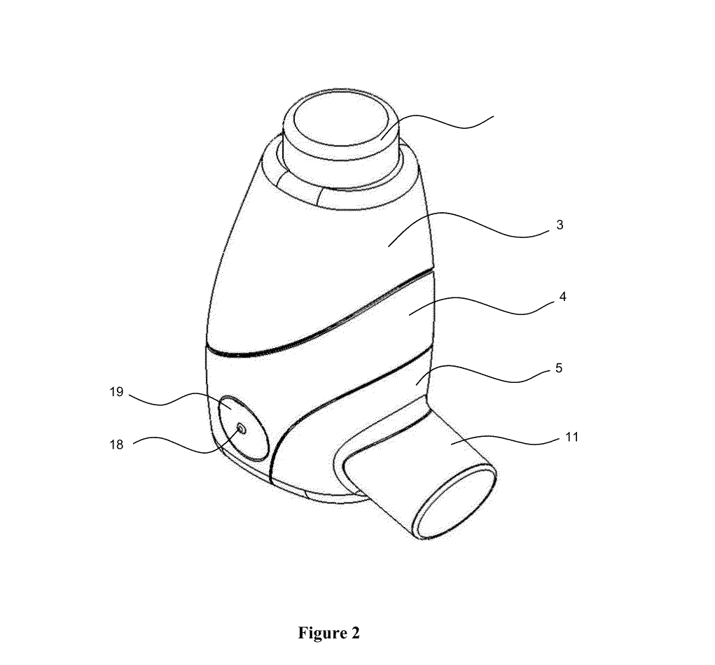

FIG. 2 is the isometric view of the device of the present invention. 1 is canister, 3 is top body part, 4 is middle body part, 5 is removable mouthpiece, 11 is mouthpiece cap, 18 is tactile pin, 19—dose counter display.

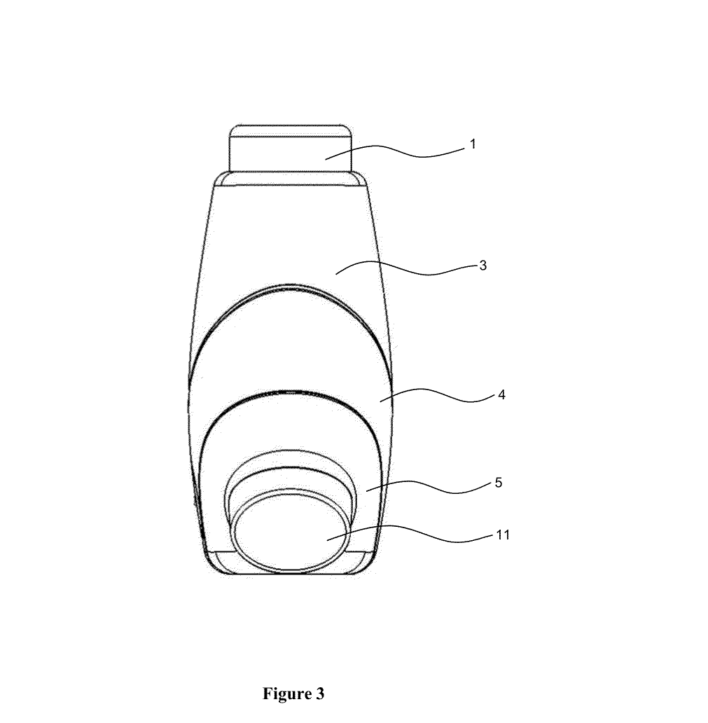

FIG. 3 is the view of the inhaler from the user end. 1 denotes canister, 3 denotes top body part, 4 denotes middle body part, 5 denotes removable mouthpiece, 11 denotes mouthpiece cap.

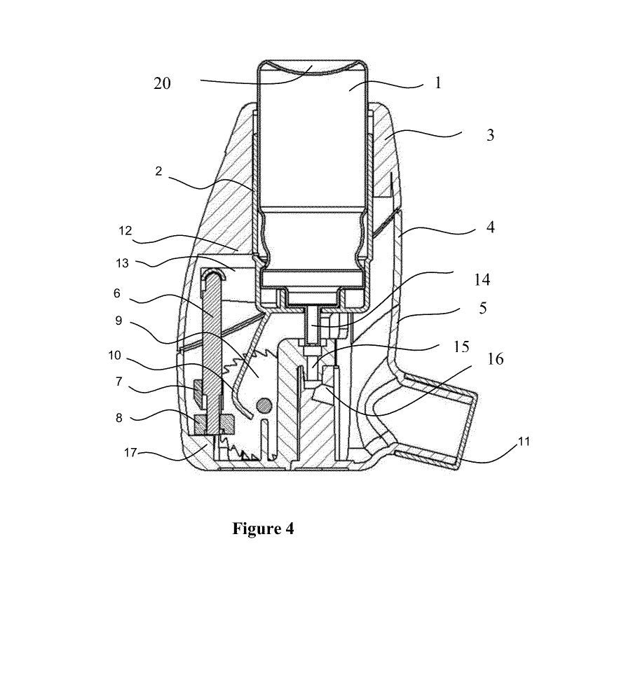

FIG. 4 represents the cross sectional side view of the device of the present invention. Where the parts of the device are numbers and are described as follows:

1—Canister; 2—Canister cap; 3—Top body part; 4—Middle body part; 5—Removable mouthpiece; 6—Pendulum rod; 7—Striker; 8—Slider; 9—Main counter gear wheel, 10—Profile spring; 11—Mouthpiece cap; 12—Canister cap movement limiter; 13—Canister cap arm; 14—Canister stem; 15—Fluid channel; 16—Nozzle; 17—Stopper feature; 20—Canister base

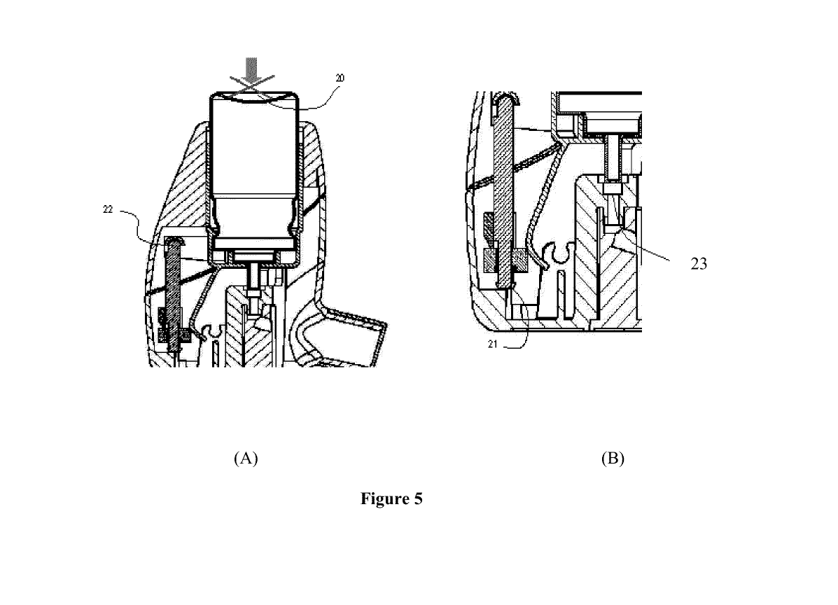

FIG. 5 (A) describes the device in the initial position. 20 denotes the canister base. 22 denotes the pivot point at which the rod is pivoted at the canister cap. FIG. 5 A indicates that initial position of the metered dose inhaler where the user cannot press the canister (indicated by a cross at the point 20.) FIG. 5 (B) is the magnified view of the device in the stage 1. Number 21 denotes the bottom of the pendulum rod, 22 denotes the pivot point and number 23 denotes upper edge of the fluid channel. The figure indicates that the pendulum rod is obstructed to sway and move downwards because of the contact between the spring profile and the slider.

The initial stages may be illustrated with the help of FIG. 5 (A). It can be described as follows: The device is vertically held in hand by the user. The pendulum rod is pivoted at the pivot point with the canister cap such that it can sway about the pivot point. The striker is free to slide on the pendulum rod along its axis vertically. The friction slider grips the pendulum rod such that external force is required to move it along the pendulum rod axis. The pendulum rod is in vertical position due to self weight and additionally weight of striker and that of slider. The slider is at a distance away from the end of the pendulum rod such that one of its side edge is touching the profile spring preventing the swaying of the pendulum rod towards the spring. The canister cap is fitted tightly with the canister such that it can move along with the canister in the vertical direction if the canister is pressed. The user is required to press the canister at its base so that the canister stem is pressed against the upper edge of fluid channel thereby dispensing a pre-metered dose of the medicament in an aerosol. The pendulum rod is prevented to move downwards by the stopper feature in the device body. As the pendulum rod is mounted at the pivot point of the canister cap, it is a part of the canister and canister cap subassembly, and because it cannot move downwards, the downward movement of the subassembly of canister and canister cap is prevented. As the movement of the whole subassembly is prevented, the canister is also locked if the user wants to press it to take a dose. Therefore the delivery of the dose cannot take place unless the canister is properly shaken. This ensures that the user is not allowed to take a dose at a condition of the device when the aerosol and the medicament are not properly mixed, for example when the device is unused for a few days.

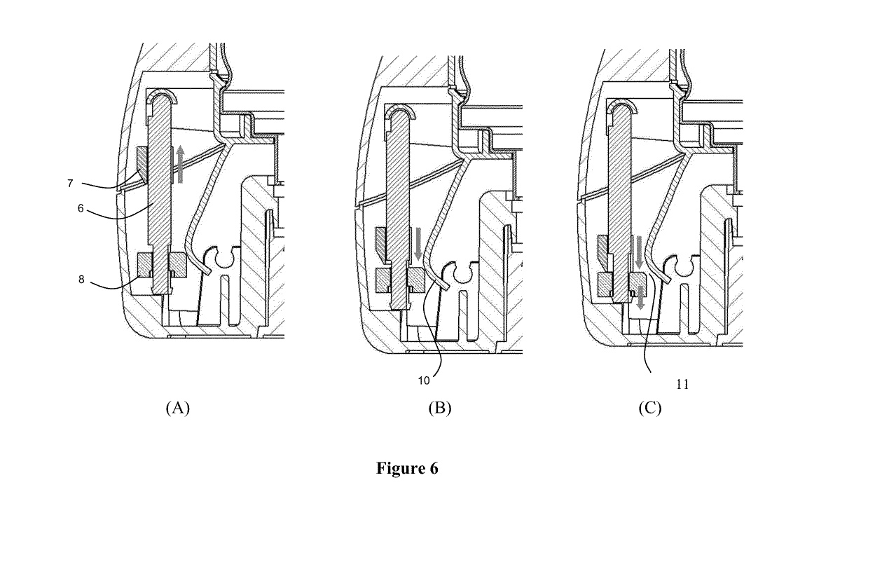

FIG. 6 (A-C) indicates the device in the next stage where the user shakes the device vigorously in hand in the up and down direction i.e along the direction of the canister axis. The device is shaken by the user. FIG. 6 (A) shows the action of striker moving to and fro over the pendulum rod and striking the slider causing it to slide towards the rod end. In FIG. 6 (A) number 6 denotes Pendulum rod, number 7 denotes striker and number 8 denotes a slider.

FIG. 6 (B) indicates the intermediate stage where the profile spring number 10 is shown.

FIG. 6 (C) shows the gap being created between the slider and the profile spring. Number 11 in the figure denotes the gap between slider and profile spring.

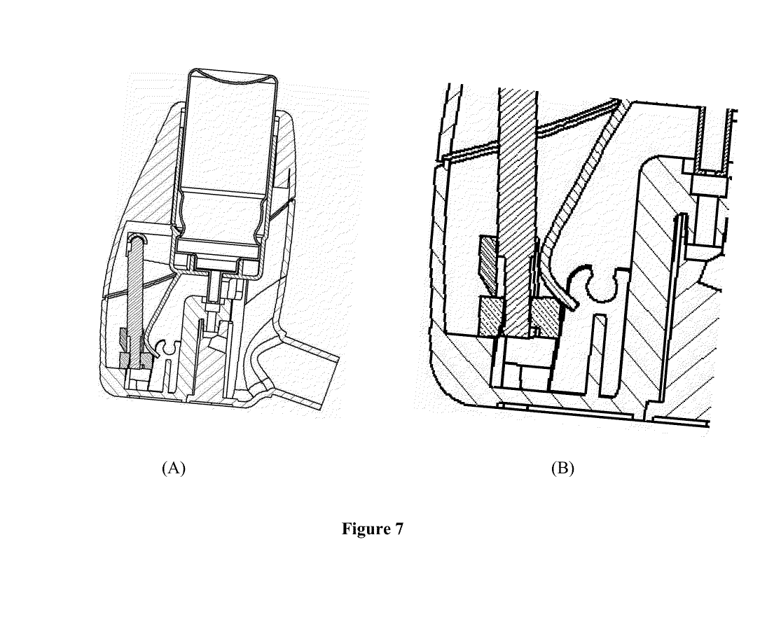

FIG. 7(A) represents the stage where the device is tilted by the user towards himself FIG. 7 (B) shows the device in stage 7 (A) in a magnified view.

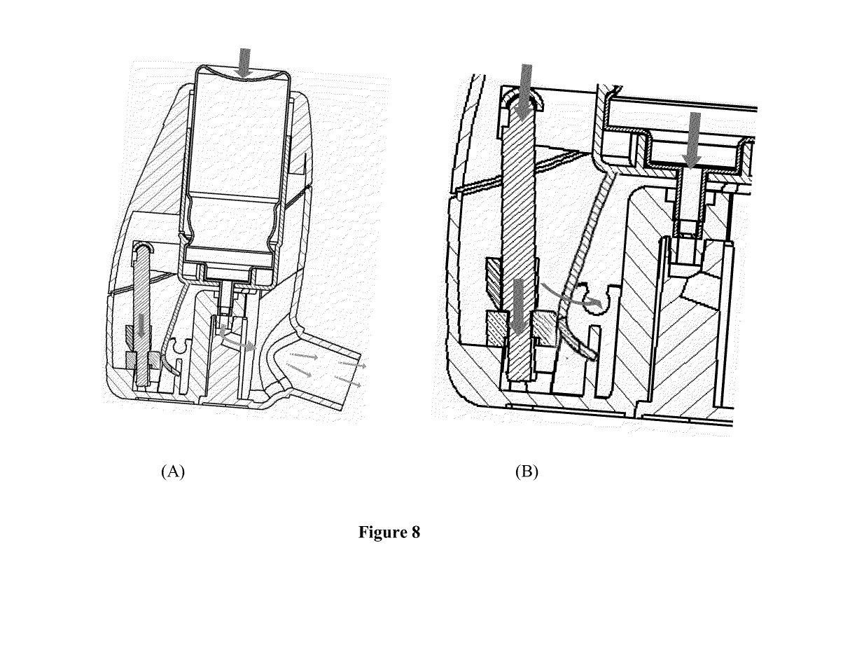

FIG. 8 (A) represents the stage of the device where the canister is pressed downwards by the user. The user presses the canister downwards to deliver the dose. The canister stem is pressed against the upper edge of the fluid channel. The dose is delivered with the propellant into the fluid channel. An aerosol spray/cloud is created through the nozzle, ejecting out through the mouthpiece which is then inhaled by the user orally. While the canister is being pressed downwards, the pendulum rod moves down and the slider is obstructed by the stopper feature in the body. This causes the relative movement between the slider and the pendulum rod and the slider is shifted to its initial position on the pendulum rod. Profile spring gets deflected towards the mouthpiece while the subassembly is moving downwards. Hence the spring is in resilience.

FIG. 8 (B) shows the device in stage 8 (A) in a magnified view.

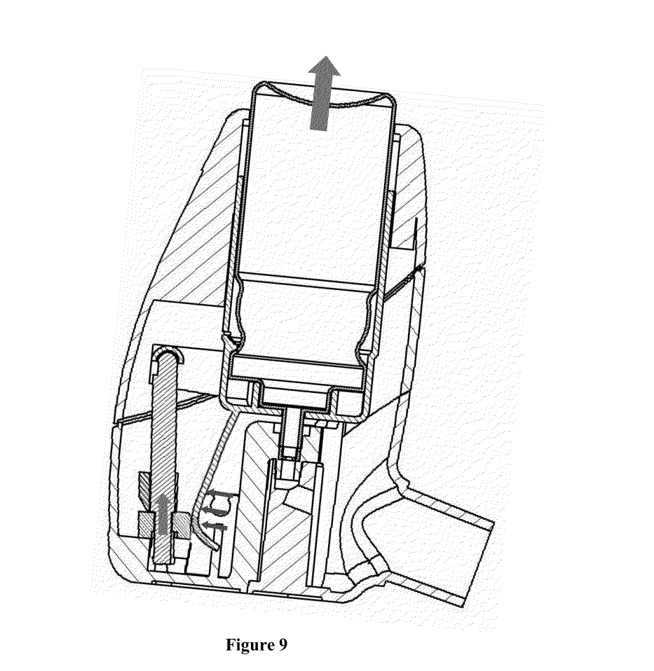

FIG. 9 represents the stage of the device where the canister is released by the user. The dose has been delivered from the mouthpiece. The user releases the canister which moves upwards due to the spring inside the canister valve stem. The profile spring which is in resilience applies force on the slider until the pendulum rod is raised upwards above the stopper feature edge. Once the pendulum rod end slips over the stopper feature the spring forces it to come to initial position as in stage 1. The slider has been reset in the initial position.

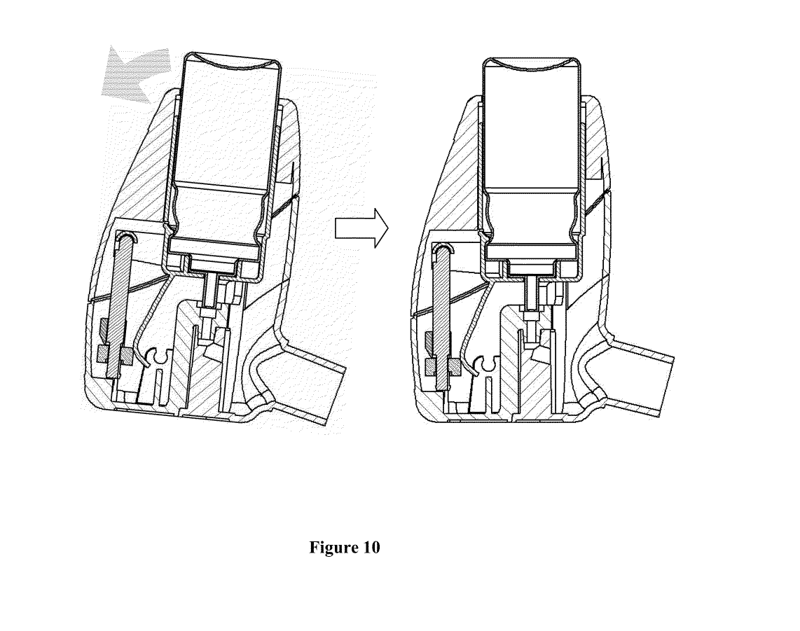

FIG. 10 represents the device being positioned from the working position to the initial position.

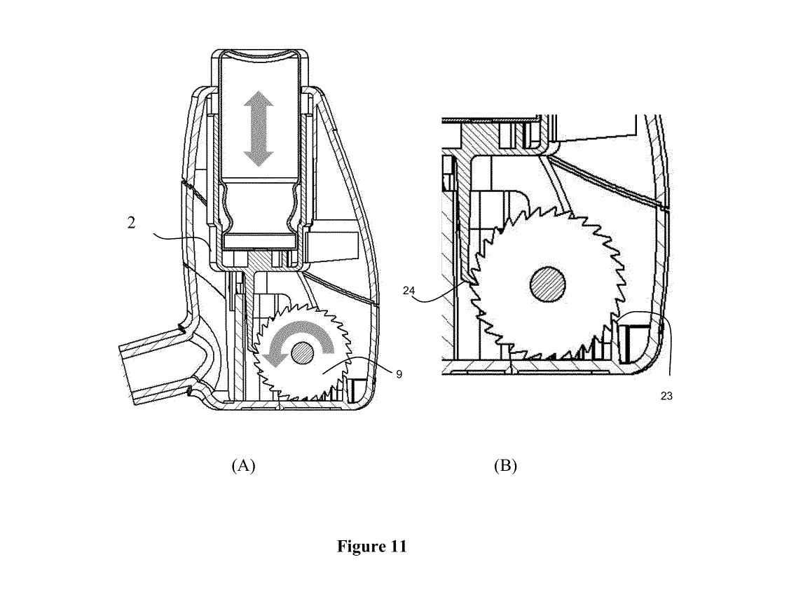

FIG. 11(A) illustrates the embodiment of the present invention, where the device includes a dose counter. In FIG. 11(A) number 2 represents canister cap, number 9 denotes main counter gear wheel, number 23 denotes stopper pawl and number 24 denotes driving pawl. The up and down movement of the canister cap is translated to unidirectional rotary movement of the main counter gear through ratchet and ratchet wheel mechanism. The reverse motion of the counter gear is arrested by the stopper pawl. Every time the canister is pressed to deliver the dose the counter gear advances by one tooth. The counter gear rotation is transmitted to the dose display through a reduction mechanism. FIG. 11 (B) shows the device in stage 11 (A) in a magnified, closer view. The dose counter display gives an idea of the remaining doses in the device and especially highlights them when the last few are remaining.

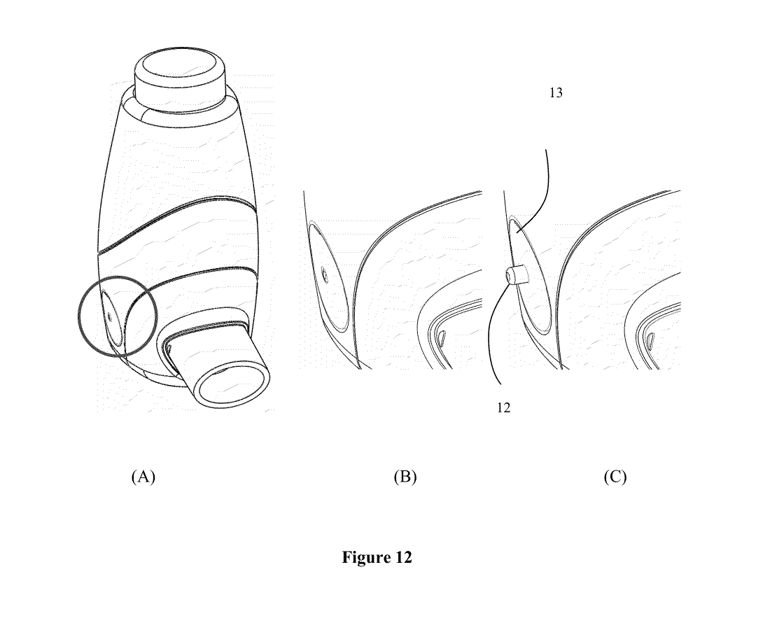

FIG. 12 illustrates the dose counter display. Number 12 denotes the tactile display and 13 denotes the visual display.

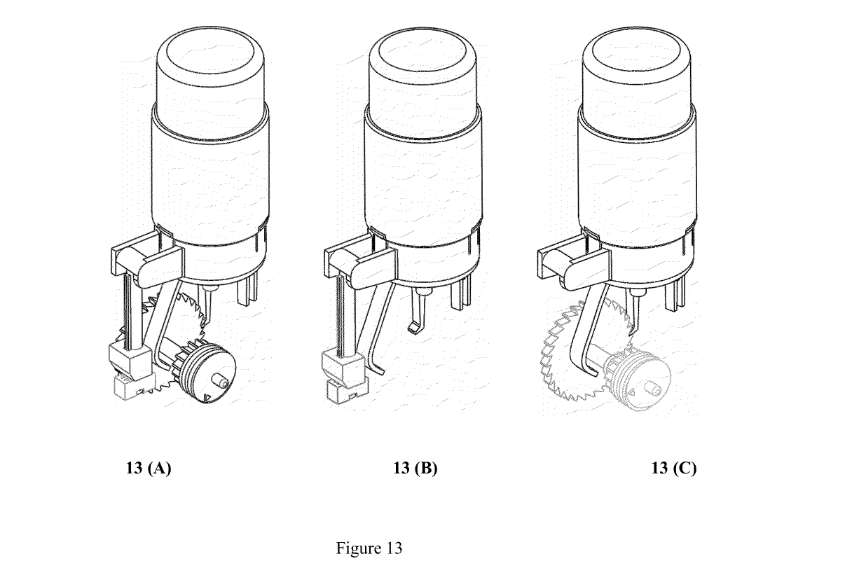

FIG. 13 (A) represents internal sub assemblies. FIG. 13 (B) represents ‘shake well’ head tilt position. FIG. 13 (C) represents counter mechanism.

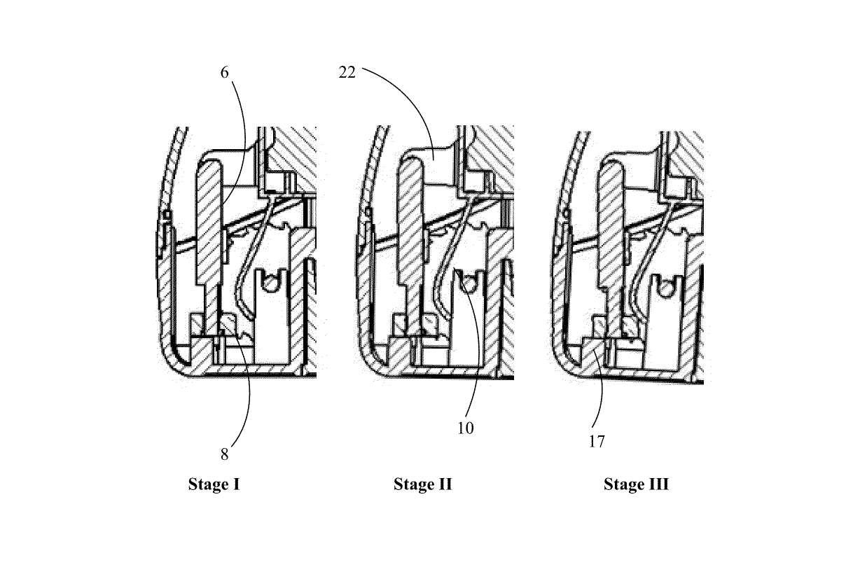

FIG. (14 A) shows the device in three stages, Stage I—an upright position when it is shaken but not tilted. The pendulum rod is vertical and the slider is at the lowest position. There exists a gap between the slider and the profile spring. The pendulum rod end is just above the stopper feature, which implies that it is obstructed by the stopper feature from coming down. In other words, the stopper 17 is obstructing the rod to move downwards and therefore the canister is still in the locked position. The stage II of FIG. 14 indicates an intermediate stage where the device is being tilted but not to the adequate position. In this case, the pendulum rod stays vertical and sways relatively to the device towards the profile spring. The distance between the slider and the profile spring is reducing. The stage III of FIG. 14 is the stage where the device is in the adequate tilted position just before the dose is being delivered. Once the device reaches this stage, the rod touches the spring allowing the rod to move towards it. In other words, the pendulum rod end is away from the stopper feature and the slider is now touching the profile spring. There is now no obstruction to the pendulum rod by the stopper feature while coming down. Since the position of the rod is moved towards the spring, the stopper no more obstruct the rod from moving downwards. This leads to unlocking of the device and therefore, the user can now press the canister to dispense the medicament. When the canister is pressed, the pendulum rod can come down, but the slider will remain in its position relative to the rod, as it will be obstructed be the stopper feature. In this way the slider will be reset to its original position relative to the pendulum rod before shaking.

### DETAILED DESCRIPTION OF THE INVENTION

The present invention provides an improved metered dose inhaler for delivery of preparation in the form of an aerosol comprising,
1. canister containing a medicament
2. actuator means
3. locking means that locks the actuator means and is unlocked upon shaking the device allowing the actuator means to deliver the medicament after it has been rendered homogeneous by shaking.

The present invention relates to an improved metered dose inhaler for delivery of an aerosol preparation comprising, canister; mouthpiece; nozzle for delivery of medicament said device characterized in that the device comprises locking means by which the device does not deliver the medicament unless the inhaler is shaken well before use. In one preferred embodiment, the metered dose inhaler of the present invention is further characterized in that the device comprises locking means by which the device does not deliver the medicament unless the inhaler is held in a correct orientation. The in built locking means may be selected from the group consisting of electronic means, mechanical means or combination thereof. The in built electronic locking means that may be used in the device are selected from the group consisting of pressure sensor, motion sensor, microprocessor, electromechanical actuator etc.

In one embodiment, the locking means used in the metered dose inhaler of the present invention comprises
a pendulum rod pivoted at the pivot point with the canister cap,
a striker which slides on the rod along its axis vertically,
spring in built with the canister cap
slider

In one embodiment, the pendulum rod is pivoted at the pivot point to the canister cap arm 13 (FIG. 4) by means of a cylindrical joint such that the pendulum rod has cylindrical protrusions that interlock into cylindrical holes in the canister cap arm. In another embodiment, the pendulum rod is pivoted at the pivot point by means of flexible ‘live hinge’ in the material of construction itself, for example polypropylene or polyethylene etc., such that the canister cap and pendulum are essentially a single body, but the cross section of the body being so low at the pivot point that the body is flexing at the pivot point and pendulum rod part of the body can sway freely due to its self weight.

In another embodiment, the pendulum rod is pivoted at the pivot point by means of hinges formed by the use of joints such as screws, pins, threads etc. The rod is in a shape such that it allows swaying of the rod with minimal friction at the pivot. On the rod are mounted a striker and a slider. The striker may be fitted on the rod by means such as slug fit or may be fitted with the help of screw or spring or by similar means. The striker generally moves on the rod in vertical direction smoothly. The striker freely slides on the pendulum rod along its axis vertically. The slider can grip the rod such that an external force is required to move it along the pendulum rod axis. The slider is referred to that part of the locking means which is placed on rod and is at a distance away from the end of the pendulum rod such that one of its side edges is touching the profile spring preventing the swaying of the pendulum rod towards the spring. The swaying of the pendulum rod occurs when the metered dose inhaler is tilted while the medicament is being administered. The swaying action of the rod allows delivery of the medicament after it has been shaken well.

According to one embodiment of the present invention, the locking means can lock the actuator means and is unlocked upon shaking the device allowing the actuator means to deliver the medicament after it has been rendered homogeneous by shaking. The minimum shaking action required to ensure correct mixing of medicament with the propellant can be defined as shaking of the device in hand by at least three times in a second by at least a distance of approximately 30 cms in the direction along the axis of the canister when assembled in the device.

In one embodiment, the weight of the striker and the weight of the slider range from about 0.15 gms to 3 gms. This weight is adjustable or tunable, depending upon the type of the suspension viscosity of the medicament, particle size of medicament, the quantum of shaking necessary for the medicament, and volume of canister etc. The striker has to strike the slider with a force of approximately at least ‘m’×‘a’ (force=m×a, where ‘m’ is the mass of the body and ‘a’ is the acceleration of the body) to overcome the friction of the slider on the pendulum rod. In one embodiment of the present invention where the mass of the striker is 0.16 gs, the force generated is the equal to the mass of striker (0.16 gms)×5×9.81 N, where 5 is considered as an approximated acceleration factor while shaking in the case of the motion of the striker and 9.81 is the acceleration due to gravity of earth at the earth's surface. This force corresponds to the force developed by the minimum shaking action to achieve the proper mixing of the medicament with the propellant.

According to one embodiment, the metered dose inhaler of the present invention may be used by the user as per following steps:
1. The user picks up the device which is held in a generally vertical position, in his palm.
2. The patient shakes the device in the direction of the axis of the canister 2-3 times by a distance of approximately at least 30 cm.
3. The patient holds the mouthpiece of the device in the mouth and tilts his head backwards along with the device.
4. The user presses the canister downwards from the canister base with his fingers. The dose is delivered.
5. The user releases the canister so that it comes to its original position.

It may be noted that the device cannot deliver the medicament unless the canister is pressed by the user. The pressing action of the canister by the user cannot take place when the device is held in the vertical position initially or it is not shaken adequately or the device is not tilted after shaking or if it is tilted without shaking it previously. As the device is locked in either of these positions, it is ensured that the user has to first shake the device, then tilt the device along with his head backwards. With these steps followed, the user is able to press the canister to deliver the dose. If such sequence of steps is not followed by the user, the canister cannot be pressed and therefore, the canister is said to be in the locked position and would not be able to dispense the medicament from the actuator.

In one most preferred embodiment, the locking means comprises a rod pivoted at the pivot point with the canister cap; a striker which slides on the pendulum rod along its axis vertically and a spring. The rod may be made up of a material like polymer or metal. The dimensions of the rod are about 35-40 mm length and 3 mm diameter approximately. The distance or the gap created between the slider and the profile spring is 1 mm to 4 mm approximately. The size of the spring is about 25-30 mm, material of construction of the spring is polymer etc. The spring is leaf spring type. In this embodiment, the device may be further locked from dispensing the medicament by prevention of pressing action of the canister bottom unless the user tilts his or her head in backward direction. Once the appropriate shaking is achieved and once the user tilts his or her head in an orientation that is desirable for optimum delivery of the medicament to the lungs i.e. about at an angle of minimum 10 degrees from the vertical in the direction such that the user's head is tilted backwards, the backward movement of the device along with the shaking of the device allows the pendulum rod to sway towards the profile spring. Both these mechanisms, therefore, ensure appropriate shaking for uniformity of the aerosol as well as an accurate, efficient head position of the user for optimum delivery of the active ingredient, once pressed.

Typically, one most preferred embodiment is represented in FIG. 4 in which the device has various parts such as canister, canister cap, a top body part, a middle body part, a optional removable mouthpiece, pendulum rod, a striker, a slider, a main counter gear wheel, a profile spring, a mouthpiece cap, a canister cap movement limiter, a canister cap arm, a canister stem, a fluid channel, a nozzle, a stopper feature, a tactile pin, a dose counter display, a canister base.

In this particular embodiment, the device functions as follows: When the device is vertically held in hand by the user, the pendulum rod is pivoted at the pivot point with the canister cap such that it can sway about the pivot point. The striker is free to slide on the pendulum rod along its axis vertically. The friction slider grips the pendulum rod such that external force is required to move it along the pendulum rod axis. The pendulum rod is in vertical position due to self weight of striker and additionally that of slider. The slider is at a distance away from the end of the pendulum rod such that one of its side edges is touching the profile spring preventing the swaying of the pendulum rod towards the spring. As the pendulum rod is mounted at the pivot point of the canister cap, it is a part of the canister and canister cap subassembly, and because it cannot move downwards, the downward movement of the subassembly of canister and canister cap is prevented. As the movement of the whole subassembly is prevented, the canister is also locked if the user wants to press it to take a dose. Therefore the delivery of the dose is thus prevented when the device, and hence, the canister is not properly shaken. This ensures that the user is not allowed to take a dose at a condition of the device when the aerosol and the medicament are not properly mixed, for example when the device is unused for a few days.

The canister cap is fitted tightly with the canister such that it can move along with the canister in the vertical direction if pressed. The user is required to press the canister at its base so that the canister stem is pressed against the upper edge of fluid channel thereby dispensing a pre-metered dose of the medicament in an aerosol. The pendulum rod is prevented to move downwards by the stopper feature in the device body. Hence the subassembly of canister and canister cap cannot move downwards and the canister stem cannot be pressed thus preventing the delivery of dose at this stage. Number 20 denotes the canister base. The device reaches the next stage where the user shakes the device vigorously in hand in the up and down direction i.e along the direction of the canister axis. The device is shaken by the user. The striker moves to and fro over the pendulum rod and strikes the slider causing it to slide towards the rod end. A gap is being created between the slider and the profile spring. In the next stage, the device is tilted by the user towards him. Because of this movement, the user has to bend his neck backwards. In the subsequent stage, the user presses the canister downwards to deliver the dose. The canister stem is pressed against the upper edge of the fluid channel. The dose is delivered with the propellant into the fluid channel. An aerosol spray or cloud is created through the nozzle, ejecting out through the mouthpiece which is then inhaled by the user orally. While the canister is being pressed downwards, the pendulum rod moves down and the slider is obstructed by the stopper feature in the body. This causes the relative movement between the slider and the pendulum rod and the slider is shifted to its initial position on the pendulum rod. Profile spring gets deflected towards the mouthpiece while the subassembly is moving downwards. Hence the spring is in resilience.

Now in the next stage of working of the device, the canister is released by the user. The dose has been delivered from the mouthpiece. The user releases the canister which moves upwards due to the spring inside the canister valve stem. The profile spring which is in resilience applies force on the slider until the pendulum rod is raised upwards above the stopper feature edge. Once the pendulum rod end slips over the stopper feature the spring forces it to come to initial position as in stage 1. The slider has been reset in the initial position.

In another embodiment, the metered dose inhaler has a dose counter. Typically, in this embodiment, there exists a main counter gear wheel, Pivot point, stopper pawl and a driving pawl. The up and down movement of the canister cap is translated to unidirectional rotary movement of the main counter gear through ratchet and ratchet wheel mechanism. The reverse motion of the counter gear is arrested by the stopper pawl. Every time the canister is pressed to deliver the dose the counter gear advances by one tooth. The counter gear rotation is transmitted to the dose display through a reduction mechanism. The dose counter display gives an idea of the remaining doses in the device and especially highlights them when the last few are remaining.

In one embodiment, the improved metered dose inhaler further comprises a breath activated mechanism. The breath activated mechanism comprises an energy storing means, a triggering means and a reset means.

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are proffered by way of example to facilitate comprehension of the invention and should not be construed to limit the scope thereof.

1. An improved metered dose inhaler for delivery of medicament in the form of an aerosol comprising,
1. canister containing a medicament
2. actuator means
3. locking means that locks the actuator means and is unlocked upon shaking the device allowing the actuator means to deliver the medicament after it has been rendered homogeneous by shaking.

2. An improved metered dose inhaler as claimed in claim 1 wherein the locking means comprises a pendulum rod pivoted at the pivot point with the canister cap, a striker which slides on the rod along its axis vertically, spring and a slider.

3. An improved metered dose inhaler as claimed in claim 2 wherein the rod is in a shape such that it allows swaying of the rod with minimal friction at the pivot.

4. An improved metered dose inhaler as claimed in claim 2 wherein the striker freely slides on the pendulum rod along its axis vertically.

5. An improved metered dose inhaler as claimed in claim 2 wherein slider grips the rod such that an external force is required to move it along the pendulum rod axis.

6. An improved metered dose inhaler as claimed in claim 2 wherein the slider is at a distance away from the end of the pendulum rod such that one of its side edge is touching the profile spring preventing the swaying of the pendulum rod towards the spring.
